(19) United States
(12) Patent Application Publication
(10) Pub. No.: US 2014/0270738 A1
(43) Pub. Date: Sep. 18, 2014

(54) Intravenous Fluid Warming Device

(71) Applicant: Michael Dean Lynch, Seattle, WA (US)

(72) Inventor: Michael Dean Lynch, Seattle, WA (US)

(21) Appl. No.: 13/843,716

(22) Filed: Mar. 15, 2013

(51) Int. Cl. A61M 5/44

(52) U.S. Cl. CPC A61M 5/445
USPC 392/443; 392/447; 392/445

(57) ABSTRACT

An apparatus and method for warming and maintaining temperature of intravenous fluid, utilizing a heater contained in a cavity of the intravenous bag. The cavity can be configured by special manufacture or by modifying available intravenous bags. The modified intravenous bag may also have insulation material for retaining heat and thermal transfer material for efficiently transferring heat from the heating component to the intravenous fluid.

### BACKGROUND OF THE INVENTION

1. Field of Invention

The present invention relates to medical devices in general and, in particular, to devices for warming intravenous (I.V.) bags and method of warming I.V. bags.

2. Background

In the use of medical equipment, intravenous fluids must be warmed up before administering to a patient. If the fluid is administered at a colder temperature to a patient, there is a serious risk of inducing hypothermia.
In a hospital, there are numerous methods used for heating up an IV bag and maintaining the temperature. Microwave ovens are commonly utilized. Other methods include heating plates and convection ovens. These methods are satisfactory for a hospital. However, all of these methods are often large, heavy, and not portable.

Military medics, paramedics, forest rangers, emergency response personnel and other people providing medical care outside of a hospital require a small, inexpensive, portable, lightweight device for heating and/or keeping intravenous fluids warm. If the fluid is not sufficiently warm when administered, the injured patient's survival chances decrease substantially before reaching the hospital.

There are portable methods available for heating I.V. and blood fluids. These methods heat the fluids in tubes external to the bag. When a patient is in need of I.V. fluids, the medical professional must exert extra effort and time unpacking and setting up a tube-based heater. These devices tend to be heavy, expensive, and require precious setup time when time is of the essence in regards to the patient. One such commercial product can take several minutes to setup. Other products can take over ten minutes of time to warm the contents of the I.V. bag, requiring the medical provider to conduct a risk analysis of hypothermia versus delaying the administration of I.V. fluids.

A field medic in the military, wilderness, or anywhere else away from the hospital has limited space to carry items and a maximum weight they can carry. Furthermore, when a patient is in immediate medical need, there is not ample time to connect extra components or heat a bag of I.V. fluid. For the forgoing reasons, there is a need for a smaller, less expensive, simpler, portable I.V. bag heater.

### SUMMARY

The present invention is directed to an apparatus and method of use that satisfies these needs. The apparatus comprises an I.V. bag containing fluid and configured in such a way as to create a cavity and a heater component that can be placed within the cavity. This apparatus is very light, takes up very little space, and is very inexpensive. In addition, the heater component can be activated far in advance of the anticipated need, whereas the I.V. fluid will maintain a safe warm temperature for an extended period of time, allowing the I.V. fluid to be ready for administration at any time without any advance notice. The cavity enclosing the heating component is enclosed by the I.V. fluid. The heating component, preferably comprising a heating element, such as a resistance wire, and a power source, such as a 9 volt disposable battery, can heat the I.V. bag all day, whereby, each day a new battery can be inserted into the power source and the I.V. bag is always ready to use. The I.V. fluid is enclosed in a second cavity.

The I.V. bag can be specially manufactured in the shape of two elliptic parabaloids, or a standard I.V. bag can be modified. Utilizing a standard I.V. bag, a portion of the fluid is removed from a full bag and one end of the bag is inverted into the other end, thereby creating a cavity. A heating component is then inserted into this cavity. When activated, the heating component will heat the I.V. bag and I.V. fluid thereby creating a safe temperature for administration of the fluid into a patient.

### BRIEF DESCRIPTION OF THE DRAWINGS

Many aspects of the invention can be better understood with reference to the following drawings. The components in the drawings are not necessarily to scale, emphasis instead being placed upon clearly illustrating the principles of the present invention. Moreover, in the drawings, like reference numerals designate corresponding parts throughout the several views.

FIG. 1 is a front view of the first embodiment of the present invention;

FIG. 2 is a front view of the external apparatus of the present invention;

FIG. 3 is a cutout view the thermal transfer material of the present invention; and

FIG. 4 is a front view of the insulation casing and of the present invention.

FIG. 5 is a perspective view of the heating component of the present invention.

FIG. 6 is a front view of the present invention with fastening means.

FIG. 7 is a front view of an I.V. bag with fluid hatch.

FIG. 8 is a front view of an I.V. bag without fluid hatch.

### DETAILED DESCRIPTION OF THE INVENTION

In the Summary of the Invention above and in the Detailed Description of the Invention, and the claims, and in the accompanying drawings, reference is made to particular features (including method steps) of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

FIG. 1 illustrates one preferred embodiment, an intravenous bag constructed of a flexible material having a top end 10 and a bottom end 12, the bag comprising a first cavity 14 and a second cavity 16, where the second cavity 16 is enclosed within the first cavity 14, as illustrated in FIG. 2. The first cavity 14 is configured to store a fluid and the second cavity 16 is configured to hold a heating component 20. Intravenous fluid 18 is hermetically contained between the first cavity 14 and the second cavity 16. The I.V. bag may be manufactured having this configuration, or may use a commonly available I.V. bag and modifying it into this configuration. The second cavity 16 encloses the heating component 20. By enclosing the entire heating component 20, the heat is efficiently transferred to the intravenous fluid 18, allowing for low power and quick heating and the ability to maintain a proper temperature for an extended period of time. The heating component 20 can be comprised of any electrical, chemical (exothermic reaction), or other kind of heating device. In this embodiment, the heating component 20 is comprised of a heating element 22, preferably a resistance wire, and a power source 24, such as an inexpensive disposable battery, a rechargeable battery, or a lithium ion battery. The preferred embodiment utilizes a disposable 9 volt battery because it is small, inexpensive, lightweight, and contains enough potential energy to power the heating element for at least one day. A medical professional can put in a battery before entering the field and the intravenous bag will stay at the proper temperature for several days or until needed. The power source 24 is preferably situated at the top 10 of the apparatus so that it can be removed, replaced, or recharged as needed. The apparatus is preferable for field medics because it adds no additional bulk and very little weight. When needing immediate warm intravenous fluid, the medical professional does not need to waste time setting up additional heating apparatus that is complicated, time-consuming, and prone to user error. The heating component may further comprise a temperature sensor and feedback device to operate at a consistent temperature.

FIG. 3 shows a thermal transfer material 26 between the second cavity 16 and the heating component 20 (the power source element of the heating component is viewable in this figure). The surface of the thermal transfer material 26 is in contact with the second cavity 16 and the heating component 20. The thermal transfer material 26 is composed of a material having attributes that increase thermal transfer efficiency between the heating component 20 and the intravenous fluid 18. In another embodiment, the second cavity 16 is constructed from thermal transfer material 26 having hermetical attributes, thereby increasing thermal transfer efficiency even further.

In another embodiment, FIG. 4, the intravenous bag is enclosed by a heat insulating casing 28. This casing 28 may be constructed from Biaxially-oriented polyethylene terephthalate, or any other thermally insulating material. The casing 28 keeps the intravenous fluid 18 warm in order to increase efficiency. The casing 28 also decreases intravenous fluid 18 heat-loss if the heating component 20 fails or the power source 24 becomes depleted. The casing 28 may cover the top end 10, bottom end 12, and/or exterior sides (exterior sides covering shown in FIG. 4.) In another embodiment, the casing 28 can be attached the thermal transfer material 26 comprising one component, thereby also preventing the second cavity 16 from inverting.

FIG. 5 illustrates the preferred embodiment of the heating component 20, having a heating element 22, in this case a resistance wire, and a power source 24, show as an alkaline battery. The power source 24 is connected to and powers the heating element 22. This particular embodiment is designed to function for at least one day and having a lightweight and inexpensive power source 24 that is replaceable quickly.

FIG. 6 illustrates a means for fastening 30 the first cavity 14 to the second cavity 16, thereby preventing the second cavity 16 from inverting. This particular means uses one or more connections to achieve the fastening means. Other fastening means can be clips, joints, seems, clasps, links, or other methods that would properly connect the first cavity 14 to the second cavity 16 to prevent the second cavity 16 from inverting. The fastening means 30 also adds stability to the invention, increasing overall durability.

A method according to the present invention for heating an intravenous liquid, comprising the steps of providing a flexible material having a first cavity 14 and a second cavity 16. The first cavity 14 and the second cavity 16, each having the preferred shape of an elliptical paraboloid. The heating component 20 is then inserted into the second cavity 16 and turned on. The first cavity 14, may enclose the second cavity 20 and may be filled with intravenous fluid 18. The top end 10 of the first cavity 14 is connected and hermetically sealed with the top end 10 of the second cavity 16. In the preferred embodiment, the heating component 20 comprising of the heating element 22 attached to the power source 24.

A method for heating an intravenous solution with a modified I.V. bag comprises the steps of providing an intravenous bag with fluid (shown in FIG. 7 and FIG. 8), removing a portion of the intravenous fluid 18, inverting the top end 10 to create the second cavity 16, and inserting a heating component 20 into the second cavity 16.

In the first step, a standard intravenous bag containing fluid available in most medical locations will suffice for the intravenous bag with fluid (FIG. 7 & FIG. 8), preferably in the one liter size. Next, a portion of the intravenous fluid 18 is removed, thereby allowing enough space to invert the top end 10, by pushing the top end 10 towards the bottom end 12, creating a cavity 16. The amount of fluid removed is approximately half the total fluid, but can be as little as approximately ¼ of the fluid or as great as approximately ¾ of the fluid. The amount of fluid removed would be determined by the actual size of the bag, the size of the heating component, the desired total size and total weight of the completed apparatus, and the anticipated need. The heating component 20 is inserted into, and completely enclosed by, the second cavity 16. The heating component may be activated in anticipation of use to allow bag to reach desired temperature in advance of administration. For instance, a medic in the military would activate each power source before leaving base, thereby allowing the intravenous bag to remain at proper temperature for at least one day.

Although the present invention has been described in considerable detail with reference to certain preferred versions thereof, other versions are possible. It is understood, therefore, that those and other modifications to the invention may be made, as might occur to one with skill in the field of this invention. Therefore, the spirit and scope of the appended claims should not be limited to the description of the preferred versions contained herein.

Therefore, having this described the invention at least the following is claimed:

1. A container for heating fluid, comprising:
a flexible material configured to define a first cavity and a second cavity,
the first cavity configured to store a fluid, and
the second cavity configured to hold a heating component,
the second cavity enclosed by the first cavity.

2. The container of claim 1 wherein the first cavity encloses the fluid.

3. The container of claim 2 wherein the heating component is insertable in the second cavity.

4. The container of claim 3 wherein the heating component comprises a heating element and a power source.

5. The container of claim 4 further comprising a temperature sensor and feedback device.

6. The container of claim 4 wherein the power source is either a disposable or a rechargeable battery.

7. The container of claim 4 wherein the power source is situated on the outer edge of the cavity for easy removal and replacement.

8. The container of claim 3 wherein a thermal transfer material is disposed between the second cavity and the heating component, the surface of the thermal transfer material is in contact with the second cavity and the heating component.

9. The container of claim 3, further comprising a heat insulating casing surrounding the first cavity.

10. The container of claim 3, wherein the second cavity is attached to the first cavity by a fastening means to prevent second cavity from inverting.

11. The container of claim 6, further comprising a heat insulating casing surrounding the first cavity.

12. A method of heating an intravenous liquid, the method comprising:
(a) providing a flexible material having a first cavity and second cavity;
(b) inserting heat component into second cavity; and
(c) providing power to the heating component to heat the intravenous solution stored in the first cavity.

13. A method of claim 12 wherein the first cavity encloses the second cavity.

14. A method of claim 13 wherein intravenous fluid is contained between the first cavity and the second cavity.

15. A method of claim 14 wherein the heat source comprises a heating element and power source.

16. A method of heating an intravenous solution with a modified I.V. bag, the method comprising:
(a) providing an intravenous bag filled with a fluid, the bag having a top end and a bottom end,
(b) removing no less than half of the fluid from the bag,
(c) inverting the top end of the bag towards the bottom end of the bag, thereby creating a cavity,
(d) inserting a heating component into the cavity.

17. The method of claim 16 wherein the heating component is activated prior to administering intravenous bag on a patient.

18. The method of claim 16, wherein the heating component comprises a heating element and a power source.

19. The heating component of claim 17 further comprising a temperature sensor and feedback device.
